(19) United States
(12) Patent Application Publication
(10) Pub. No.: US 2007/0050925 A1
(43) Pub. Date: Mar. 8, 2007

(54) Composition and method for delivering infrared radiation and ionically charged particles to hair

(76) Inventor: Farouk M. Shami, Woodlands, TX (US)

Correspondence Address: BRACEWELL & GIULIANI LLP, P.O. BOX 61389, HOUSTON, TX, 77208-1389, US

(21) Appl. No.: 11/511,118

(22) Filed: Aug. 28, 2006

Related U.S. Application Data
(60) Provisional application No. 60/711,829, filed on Aug. 26, 2005.

(51) Int. Cl. A61K 8/64

(52) U.S. Cl. 8/406; 424/70.14

(57) ABSTRACT

A composition and method for use in hair care and treatment. The composition of the present invention preferably includes a ceramic material and a hair preparation, whereby the composition delivers infrared radiation and ionic molecules to the hair when the composition is applied to the hair. A method of using the composition includes the step of applying the composition to the hair. A method of making the composition includes the steps of forming the one or more ceramic or non-ceramic materials into a powder or into fine particles and mixing the powder or fine particles with the hair preparation.

### CROSS-REFERENCE TO RELATED APPLICATIONS

This nonprovisional application claims the benefit of provisional application Ser. No. 60/711,829, filed Aug. 26, 2005, which is hereby incorporated by reference in its entirety.

### BACKGROUND OF THE INVENTION

1. Field of the Invention

The present invention relates generally to the care and treatment of hair, and in particular to a composition and method for hair care and treatment that utilizes ceramic materials together with hair preparations to deliver infrared radiation and/or ionically charged particles to hair.

2. Description of the Related Art

It is known that human hair can become electrostatically charged, either naturally or as a result of brushing or combing. Electrostatically charged hair can be unruly, unmanageable and difficult to treat or style.

It is recognized that delivering ionically charged molecules, preferably negative ions, to the hair can reduce the level of electrostatic charge in the hair. Negative ions are molecules with an extra electron, and are believed to have a positive, cleansing effect upon the atmosphere. Various types of hair care and styling equipment, for example hair dryers and hair rollers, have been utilized to deliver these negative ions to hair. For example, certain hair dryers have an ionizing device contained within the dryer for dispensing ions into the heated air flow exiting the dryer.

It is also known that hair dryers are capable of delivering infrared radiation to the hair. Infrared radiation is generally described as radiation falling between visible light and microwave radiation in the electromagnetic spectrum, and is believed to have beneficial properties for hair, including but not limited to, reducing drying time for wet hair. Certain hair dryers utilize a ceramic material located inside the hair dryer to produce infrared radiation that is delivered to the hair in the heated air stream emitted from the dryer.

It would be advantageous to provide a composition and method for delivering negative ions and/or infrared radiation to the hair with increased uniformity and deeper penetration of the negative ions and/or infrared radiation into the hair. It would also be advantageous to provide a composition and method for hair care and treatment that delivers negative ions and infrared radiation to the hair and that is incorporated with conventional hair care preparations instead of the aforementioned hair care and styling equipment.

Accordingly, prior to the development of the present invention, there has been no composition and method for hair care and treatment that: delivers negative ions and infrared radiation to the hair via a hair care preparation; reduces the potential for heat damage to the hair; delivers negative ions and/or infrared radiation to the hair uniformly and in a deeply penetrating manner; is capable of improving the manageability of the hair; and is economical and easy to apply and/or use. Therefore, the art has sought a composition and method for hair care and treatment that delivers negative ions and infrared radiation to the hair via a hair care preparation which: improves manageability of the hair and reduces the potential for heat damage; is capable of deeply penetrating and uniformly treating the hair; and is economical and easy to apply and/or use.

### SUMMARY OF THE INVENTION

In accordance with the invention, the foregoing advantages have been achieved through the present composition for use in delivering negative ions and/or infrared radiation to hair. The composition of the present invention preferably includes a ceramic material combined with one or more hair preparations, whereby these components are capable of being combined in an aqueous or non-aqueous based solution, and whereby the ceramic material delivers negative ions and/or infrared radiation to hair. In an embodiment of the invention, the hair preparation is a hair colorant, such that the composition both colors hair and delivers negative ions and/or infrared radiation to hair.

In accordance with another aspect of the invention, the foregoing advantages have also been achieved through the present method for delivering negative ions and/or infrared radiation to hair. This aspect of the present invention preferably includes the steps of applying a hair preparation solution to the hair, whereby the solution includes a ceramic material combined with one or more hair preparations, and whereby these components are capable of being combined in an aqueous or non-aqueous based solution, and delivering negative ions and/or infrared radiation to hair. In an embodiment of the invention, the hair preparation is a hair colorant, such that the method can be used both to color hair and deliver negative ions and/or infrared radiation to hair. The method can further include the step of heating the composition to enhance to delivery of negative ions and infrared radiation to the hair.

In accordance with still another aspect of the invention, the foregoing advantages have also been achieved through the present method for forming a hair preparation composition for use in delivering negative ions and/or infrared radiation to hair. The method preferably includes the steps of forming the ceramic or non-ceramic materials into a powder or into fine particles and mixing the powder or fine particles with one or more hair preparations in an aqueous or non-aqueous based solution.

The compositions and methods of the present invention, when compared with previously proposed compositions and methods for hair care and treatment, have the advantages of: protecting the hair and improving the vibrancy and shininess of the hair; providing more uniform distribution and deeper penetration of infrared radiation and/or negative ions into the hair; decreasing the time required for processing and drying hair; conditioning the hair; and being economical and easy to apply and/or use.

While the invention will be described in connection with the preferred embodiments, it will be understood that it is not intended to limit the invention to those embodiments. On the contrary, it is intended to cover all alternatives, modifications, and equivalents as may be included within the spirit and scope of the invention as defined by the claims.

### DETAILED DESCRIPTION OF THE INVENTION

The present invention involves a hair preparation composition and methods for forming and using said composition.

In an embodiment of the present invention, the hair preparation composition includes one or more ceramic materials combined with one or more hair preparation products. Ceramics are generally defined as inorganic materials that are typically crystalline in nature. Examples of ceramic materials include, but are not limited to, silicon oxide, aluminum oxide and iron oxide.

The ceramic materials in the hair preparation preferably deliver ionically charged molecules and/or infrared radiation to the hair, which results in, for example, improved manageability, color fastness and/or drying capability to the hair.

The hair preparation in the composition of the present invention can be, for example, shampoo, conditioner, bleach, color or hair colorants, gel, mousse, spray, rinse, lotion or other like products, or combinations thereof. In general, the hair preparation can be any hair product that can be applied directly to the recipient's hair such that the preparation contacts the hair, and the ionically charged molecules and/or infrared radiation are delivered to the strands of hair uniformly and in a deeply penetrating manner.

In an embodiment of the present invention, the hair preparation composition is applied directly to the recipient's hair, whereby the preparation forms a thin coating over the hair strands. The delivery of the ionically charged molecules and/or infrared radiation to the hair is initially activated or enhanced by the natural heat from the recipient's head. Delivery can be further activated, or enhanced, if desired, by the use of a hair dryer or other similar heat supplying equipment.

In another embodiment of the present invention, the preparation can be applied to the recipient's hair, and delivery of the infrared radiation and/or ionically charged molecules can be initially activated, or enhanced, by applying heat from a hair dryer or other similar equipment to the coated hair strands.

Delivery of the infrared radiation and/or ionically charged molecules to the hair can also be activated, or enhanced, by chemicals or other components in, or added to, the hair preparation such as, for example, hydrogen peroxide.

In an embodiment of the present invention, wherein the hair preparation is a hair colorant, it has been discovered that the hair preparation composition advantageously improves penetration of the color deposit in the hair, or strands of hair, and traps the coloring on the hair. Preferably, the composition slightly raises, but does not harshly open, the cuticle layer of the hair. As a result, the hair coloring cannot be easily rinsed out after multiple washings, and the color does not fade as quickly. It has been discovered that the composition of the present invention allows the hair to retain and maintain its color for longer periods of time, with reduced damage to the hair and a significant improvement in color maintenance properties after multiple washings.

According to an embodiment of the present invention, the ceramic material is combined with a hair preparation to form a hair preparation composition with improved properties. A preferred embodiment of the present invention, for a hair colorant, contains permanent hair dye and a ceramic material. Other compositions and embodiments of the present invention other than the composition shown above may exist. For example, the composition may also contain additional elements, color bases, intermediate dyes, color molecules, or an alkaline agent such as, for example, monoethanolamine. Natural silk can also be added to the composition. The natural silk provides additional moisturizing and conditioning to the hair strands and protects the hair during the coloring process.

In a preferred embodiment, the ceramic material can include one or more of the elements from the group consisting of calcium, aluminum, silicon, chromium, titanium, iron, phosphorus, manganese, lanthanum, potassium, cerium, magnesium, boron and vanadium. In an embodiment, the approximate composition of each element present in the ceramic material is at least 0.001%. In another embodiment, the approximate composition of each element present in the ceramic material is at least 5.0%. In another embodiment, each of the one or more elements present in the ceramic material comprise in the range from 0.001-5.0% of the composition. Other combinations of elements and/or other compositions may also be utilized. For example, oxides of certain of these elements may be utilized in certain embodiments of the invention. Also, the ceramic material may comprise or include one or more of these elements mixed with other infrared radiation producing materials. It is to be noted that the composition of the present invention can vary depending upon the desired results and the different types of preparations, such as shampoos, conditioners, dyes, hair colorants and the like.

Preferably, if the hair preparation composition contains a hair colorant, the composition according to the present invention will oxidize a greater number of molecules in the hair colorant when the composition is mixed with a developer such as, for example, hydrogen peroxide. As a result, the treated hair will display, among other advantageous characteristics, stronger and longer lasting color as well as a more permanent shine and reduced damage.

The hair preparation composition of the present invention can be ammonia-based or nonammonia-based. In a preferred embodiment, however, the hair preparation composition is ammonia free in order to maintain a relatively constant pH. Further, the composition of the present invention may be utilized in either water-based or anhydrous, nonwater-based hair preparations. Anhydrous systems are utilized, for example, when chemicals which are reactive with water, such as potassium persulfate, are present in the hair preparation. According to an embodiment of the present invention, isododecane is utilized as a preferred solvent in an anhydrous system.

In another embodiment of the present invention, one or more non-ceramic materials can be combined with one or more hair preparations. Examples of such non-ceramic materials include, but are not limited to, minerals such as mica, muscovite, tourmaline and lanthanum. These and other non-ceramic materials can also produce infrared radiation and/or negative ions for distribution in the recipient's hair. Also, a combination of ceramic and non-ceramic materials can be added to one or more hair preparations in order to deliver the desired amount of infrared radiation and/or negative ions to the hair strands.

A method for treating hair using the hair preparation composition according to the present invention includes the step of applying the hair preparation composition to the hair of a recipient, such that the hair strands are coated with the hair preparation composition. The hair preparation product preferably forms a film or coating on the hair strands, which protects the hair from damage, improves the manageability and drying properties in the hair, deeply penetrates into the hair and purifies and/or conditions the hair. The composition also nourishes the scalp and stimulates circulation to promote thicker, fuller hair in an embodiment. Further, the composition deeply penetrates into the hair relatively quickly, which reduces the likelihood of damage caused from excessive over-drying. The hair preparation composition can be left in the hair or rinsed out of the hair, depending, for example, on the particular preparation product used in the composition. Also, additional steps can be performed, if necessary, to activate the delivery of infrared radiation and/or negative ions to the hair strands or to trigger the hair coloring process.

A method of producing the hair preparation composition according to the present invention includes the step of adding a ceramic or non-ceramic material to one or more hair preparation products, whereby when the composition is applied to a recipient's hair, infrared radiation and/or negative ions are delivered to the hair and whereby the hair thereafter exhibits improved manageability and styling properties. In one embodiment, the method of the present invention can include the steps of forming the one or more ceramic or non-ceramic materials into a powder or into fine particles, and then mixing the powder or fine particles with the one or more hair preparations.

It is to be understood that the invention is not limited to the exact details of construction, operation, exact materials, or embodiment shown in described, as obvious modifications and equivalents will be apparent to one skilled in the art, or technological field. Accordingly, the invention is therefore to be limited only by the scope of the appended claims.

1. A composition for use in coloring hair and improving color retention properties in hair, the composition comprising:
hair colorant; and
a ceramic material, whereby the ceramic material delivers infrared radiation and negative ions to the hair when the composition is applied to the hair.

2. The composition of claim 1, further comprising natural silk.

3. The composition of claim 1, whereby the composition is ammonia-free.

4. The composition of claim 1, whereby the ceramic material includes at least one element from the group consisting of calcium, aluminum, silicon, chromium, titanium, iron, phosphorus, manganese, lanthanum, potassium, cerium, magnesium, boron and vanadium.

5. The composition of claim 4, whereby each of the one or more elements present in the ceramic material comprise in the range from 0.001-5.0% of the composition.

6. The composition of claim 1, wherein the hair colorant and ceramic material are combined in a non-aqueous based solution.

7. A hair care composition comprising:
hair preparation; and
a ceramic material, whereby the ceramic material delivers infrared radiation and negative ions to the hair when the hair care composition is applied to the hair.

8. The composition of claim 7, wherein the hair preparation comprises at least one member of the group consisting of shampoo, conditioner, bleach, gel, mousse, spray, rinse and lotion.

9. The composition of claim 7, whereby the ceramic material includes at least one element from the group consisting of calcium, aluminum, silicon, chromium, titanium, iron, phosphorus, manganese, lanthanum, potassium, cerium, magnesium, boron and vanadium.

10. The composition of claim 7, whereby each of the one or more elements present in the ceramic material comprise in the range from 0.001-5.0% of the composition.

11. The composition of claim 7, wherein the hair preparation and ceramic material are combined in a non-aqueous based solution.

12. A method for coloring hair and delivering negative ions and infrared radiation to hair, the method comprising the step of applying the composition of claim 1 to the hair.

13. The method of claim 12, further comprising the step of heating the composition to enhance the delivery of negative ions and infrared radiation to the hair.

14. The method of claim 12, further comprising the step of adding a developer to the composition to enhance the delivery of negative ions and infrared radiation to the hair.

15. The method of claim 14, wherein the developer comprises hydrogen peroxide.

16. A method for delivering negative ions and infrared radiation to hair, the method comprising the step of applying the hair care composition of claim 7 to the hair.

17. The method of claim 16, further comprising the step of heating the composition to enhance the delivery of negative ions and infrared radiation to the hair.

18. The method of claim 16, further comprising the step of adding a developer to the composition to enhance the delivery of negative ions and infrared radiation to the hair.

19. The method of claim 16, wherein the developer comprises hydrogen peroxide

20. A method of forming a hair preparation composition for use in delivering negative ions and/or infrared radiation to hair, the method comprising the steps of:
forming a ceramic material into a powder; and
mixing the powder with at least one hair preparation to form a solution.
